(19) United States
(12) Patent Application Publication
(10) Pub. No.: US 2024/0019429 A1
(43) Pub. Date: Jan. 18, 2024

(54) METHODS AND RELATED ASPECTS FOR DETERMINING BINDING KINETICS OF LIGANDS

(71) Applicant: ARIZONA BOARD OF REGENTS ON BEHALF OF ARIZONA STATE UNIVERSITY, Scottsdale, AZ (US)

(72) Inventors: Shaopeng WANG, Chandler, AZ (US); Guangzhong MA, Tempe, AZ (US); Xiaoyan ZHOU, Tempe, AZ (US)

(73) Assignee: ARIZONA BOARD OF REGENTS ON BEHALF OF ARIZONA STATE UNIVERSITY, Scottsdale, AZ (US)

(21) Appl. No.: 18/349,650

(22) Filed: Jul. 10, 2023

Related U.S. Application Data
(60) Provisional application No. 63/388,034, filed on Jul. 11, 2022.

(51) Int. Cl. G01N 33/557; G01N 33/543; G01N 21/552

(52) U.S. Cl. CPC G01N 33/557; G01N 33/54373; G01N 21/553

(57) ABSTRACT

Provided herein are methods of determining binding kinetics of a ligand. In some embodiments, the methods include contacting the ligand with a first surface of a substrate, which first surface comprises an electrically conductive coating and a population of receptors connected to the first surface via one or more linker moieties, wherein the receptors bind, or are capable of binding, to the ligand, applying an alternating current electric field to the substrate to induce the receptors to oscillate proximal to the first surface of the substrate, and detecting changes in oscillation amplitudes of the receptors over a duration. Related receptor oscillator array devices, systems and computer readable media are also provided.

### CROSS-REFERENCE TO RELATED APPLICATIONS

This application claims the benefit of U.S. Provisional Patent Application Ser. No. 63/388,034 filed Jul. 11, 2022, the disclosure of which is incorporated herein by reference.

### STATEMENT OF GOVERNMENT SUPPORT

This invention was made with government support under R33 CA235294 awarded by the National Institutes of Health. The government has certain rights in the invention.

### REFERENCE TO ELECTRONIC SEQUENCE LISTING

The application contains a Sequence Listing which has been submitted electronically in .XML format and is hereby incorporated by reference in its entirety. Said .XML copy, created on Jul. 10, 2023, is named “0391.0053.xml” and is 2.14 kilobytes in size. The sequence listing contained in this .XML file is part of the specification and is hereby incorporated by reference herein in its entirety.

### BACKGROUND

Molecular interactions are closely related to many aspects of cell biology, such as cell signaling, enzymatic reactions, early diagnosis of diseases, and drug screening. Mainstream label-free techniques such as surface plasmon resonance (SPR) and biolayer interferometry (BLI) quantify the interactions by measuring the surface refractive index or mass. Although the principle is simple and straightforward, these techniques are fundamentally insensitive to small molecules, as the sensitivity diminishes with mass. On the other hand, small molecule interaction is extremely important in biological systems and drug discovery industry. For example, dozens of FDA approved drugs are in the form of small molecule every year. There is an immediate need to improve the sensitivity of the current label-free techniques.

### SUMMARY

This disclosure describes receptor oscillator arrays, systems, computer readable media, and related methods for determining binding kinetics of ligands with receptors. In some embodiments, for example, the methods and related aspects of the present disclosure address pre-existing challenges of measuring ligand-receptor binding activities via the use of receptor oscillators. These and other aspects will be apparent upon a complete review of the present disclosure, including the accompanying figures.

In one aspect, the present disclosure provides a method of determining binding kinetics of a ligand (e.g., an antibody, a small molecule, or the like). The method includes contacting the ligand with a first surface of a substrate, which first surface comprises an electrically conductive coating and a population of receptors connected to the first surface via one or more linker moieties, wherein the receptors bind, or are capable of binding, to the ligand, inducing the receptors to oscillate proximal to the first surface of the substrate, and detecting changes in oscillation amplitudes of the receptors over a duration, thereby determining the binding kinetics of the ligand.

In some embodiments, the inducing step comprises applying an alternating current electric field to the substrate. In some embodiments, the detecting step comprises separating a detectable signal received from the receptors over the duration into a direct current component and an alternating current component. In some embodiments, the alternating current electric field is applied to the substrate using an electrode system that comprises a reference electrode, a counter electrode, and a working electrode. In some embodiments, the method includes determining a change in mass of one or more of the receptors from a detected reflectivity change of a surface of the substrate. In some embodiments, the method includes determining a change in charge of one or more of the receptors from a detected oscillation amplitude change of the one or more of the receptors. In some embodiments, the linker moieties comprise polymers. In some embodiments, the receptors comprise a charge. In some embodiments, the method includes determining size, charge, and/or conformation alterations of one or more of the receptors from the changes in oscillation amplitudes.

In some embodiments, the method includes detecting the changes in the oscillation amplitudes of the receptors using a plasmonic imaging technique and/or a microscopic imaging technique. In some embodiments, the electrically conductive coating comprises gold (Au), indium tin oxide (ITO), silver (Ag), copper (Cu), and/or aluminum (Al). In some embodiments, the linker moieties comprise polyethylene glycol (PEG) moieties and/or biomolecules (e.g., a nucleic acid, a protein, or the like). In some embodiments, one or more spacer moieties are connected to the first surface and/or to the linker moieties. In some embodiments, the method includes quantifying the binding kinetics and binding affinity of the ligand using the detected changes in the oscillation amplitudes of the receptors over the duration. In some embodiments, the method includes determining the binding kinetics of the ligand in substantially real-time.

In some embodiments, the detecting step comprises introducing an incident light toward a second surface of the substrate to induce a plasmonic wave at least proximal to the first surface of the substrate and detecting a change in intensity of the incident light reflected at an interface of the first surface of the substrate. In some embodiments, the method includes introducing the incident light via at least one objective lens and/or at least one prism. In some embodiments, the method includes introducing the incident light using a superluminescent diode (SLED), a laser and/or a light emitting diode (LED). In some embodiments, the method includes detecting the changes in the oscillation amplitudes of the receptors over the duration using a CMOS camera.

In another aspect, the present disclosure provides a receptor oscillator array device, comprising a substrate that comprises a first surface that comprises an electrically conductive coating and a population of receptors connected to the first surface via one or more linker moieties, wherein the receptors bind, or are capable of binding, to a ligand. In some embodiments, the electrically conductive coating comprises gold (Au), indium tin oxide (ITO), silver (Ag), copper (Cu), and/or aluminum (Al). In some embodiments, the linker moieties comprise polyethylene glycol (PEG) moieties and/or biomolecules (e.g., a nucleic acid, a protein, or the like). In some embodiments, one or more spacer moieties are connected to the first surface and/or to the linker moieties.

In another aspect, the present disclosure provides a system for determining binding kinetics of a ligand. The system includes a substrate having a first surface and a second surface opposite the first surface, wherein the first surface comprises an electrically conductive coating and a population of receptors connected to the first surface via one or more linker moieties, wherein the receptors bind, or are capable of binding, to the ligand, a power source electrically connected to the substrate, which power source is configured to apply an alternating current electric field to the substrate, and an objective lens or a prism disposed proximal to the second surface of the substrate. The system also includes a light source configured to introduce light through the objective lens or the prism to induce a plasmonic wave at least proximal to the first surface of the substrate, and a detector configured to collect light reflected from the substrate. In addition, the system also includes a controller that comprises, or is capable of accessing, computer readable media comprising non-transitory computer-executable instructions which, when executed by at least one electronic processor, perform at least: applying an alternating current electric field to the substrate to induce the receptors to oscillate proximal to the first surface of the substrate using the power source; introducing an incident light toward the second surface of the substrate from the light source to induce the plasmonic wave at least proximal to the first surface of the substrate; and detecting changes in oscillation amplitudes of the receptors over a duration to thereby determine the binding kinetics of the ligand.

In another aspect, the present disclosure provides a computer readable media comprising non-transitory computer executable instruction which, when executed by at least electronic processor, perform at least: applying an alternating current electric field to a substrate having a first surface and a second surface opposite the first surface, wherein the first surface comprises an electrically conductive coating and a population of receptors connected to the first surface via one or more linker moieties, wherein the receptors bind, or are capable of binding, to a ligand, which alternating current electric field induces the receptors to oscillate proximal to the first surface of the substrate using the power source; introducing an incident light toward the second surface of the substrate from the light source to induce the plasmonic wave at least proximal to the first surface of the substrate; and detecting changes in oscillation amplitudes of the virions over a duration to thereby determine the binding kinetics of the ligand.

In some embodiments of the system or computer readable media, the electrically conductive coating comprises gold (Au), indium tin oxide (ITO), silver (Ag), copper (Cu), and/or aluminum (Al). In some embodiments of the system or computer readable media, the linker moieties comprise polyethylene glycol (PEG) moieties and/or biomolecules (e.g., a nucleic acid, a protein, or the like). In some embodiments, one or more spacer moieties are connected to the first surface and/or to the linker moieties. In some embodiments of the system or computer readable media, the non-transitory computer-executable instructions which, when executed by the electronic processor, further perform at least: quantifying the binding kinetics and binding affinity of the ligand using the detected changes in the oscillation amplitudes of the receptors over the duration.

### BRIEF DESCRIPTION OF DRAWINGS

FIG. 1 is a flow chart that schematically shows exemplary method steps of determining binding kinetics of a ligand according to some aspects disclosed herein.

FIG. 2 is a schematic diagram of an exemplary system suitable for use with certain aspects disclosed herein.

FIGS. 3A-3F. Detection principle of SPR-OBL. (a) The oscillating biolayer (OBL) is fabricated on a gold film surface. A sinusoidal potential is applied to drive the OBL into oscillation by a three-electrode system, where an Ag/AgCl wire, a Pt coil and the gold surface serve as the reference, counter and working electrode, respectively. A drug delivery system is used to flow samples. The oscillation of biolayer is monitored by a plasmonic imaging setup. (b) A zoom-in picture showing the scheme of the OBL, which consists of a protein receptor layer connected to the surface via a flexible polymer cushion. The receptors are charged in solution, thus can oscillate with the applied electric field. Binding of ligand to the receptor alters the size, charge, and conformation of the receptor, and hence the oscillation. (c) The binding and dissociation of the ligands lead to mass and charge change to the OBL. The DC and AC component of the OBL signal are separated, which arise from the surface reflectivity (mass) change and oscillation amplitude (charge) change, respectively. (d) A SPR image showing an OBL functionalized region and an adjacent background region without OBL. (e) The upper panel shows the relationship between the oscillation, background charging and applied potential at a frequency of 5 Hz. The bottom panel is the FFT of the biolayer oscillation signal and background charging, showing a pronounced peak at 5 Hz. The height of the peak is proportion to the amplitude of oscillation. (f) Oscillation response to the applied potential. The oscillation amplitude determined from the OBL region and the background region in c are plotted vs. potential. The difference between the two curves represents the mechanical response of the OBL.

FIGS. 4A-4C. Validation of the principle. (a) Streptavidin OBL response to different concentrations of NaCl solution. The plot shows the raw data collected in an OBL region and a background region. The inset is a zoom-in of the marked section, showing the oscillation. Applied potential: 0.3 V, 5 Hz. Shown in the middle of the figure, 0.1-Hz low pass filter is applied to the OBL signal in a to extract the DC signal, which reflects the refractive index change of the medium. At the bottom, a 5-Hz bandpass fast Fourier transform filter is applied to the OBL data in a every 1 second to filter out the AC component. The AC signal reflects the charge change of the OBL. The greyscale shaded line is a guide to the eye. (b) and (c) The SPR-OBL signal and fluorescence signal of FITC-streptavidin in response to applied potential at pH 7.4 and pH 3.4, respectively. The top images depict the oscillation status of the OBL at the position marked by the dotted line in the bottom panels.

FIGS. 5A-5C. Detection of large biomolecule. (a) BSA binding to anti-BSA. Both mass and charge of the OBL will change, which leads to surface reflectivity and oscillation amplitude change. (b) The DC response (black dots) was fitted to the first order of kinetics (lighter greyscale curve). (c) The AC response (black dots) and fitting (lighter greyscale curve).

FIGS. 6A-6G. Measuring the binding kinetics of small molecule ligand to membrane protein. (a) Biotinylated KscA-Kv1.3 nanodisc was immobilized on streptavidin OBL via biotin-streptavidin bonding. Two different small molecules, compound 1 (320 Da) and PAP-1 (350 Da), were flowed over the OBL for the measurement. (b) OBL AC response for compound 1 at 25° C. The solid curves are fitting of the data to the first-order binding kinetics. (c) The compound 1 binding was measured again under 30° C. (d) Control experiment of compound 1 binding to the streptavidin OBL at 25° C. (e) AC response curves for PAP-1 binding to the nanodisc at 25° C., the solid lines are fittings of the data. (f) The interaction in e was determined under (g) Control experiment of PAP-1 by using a streptavidin OBL.

FIGS. 7A-7E. Ion-protein interaction. (a) Schematic showing the charge and conformation change of calmodulin caused by Ca²⁺ binding. (b) AC response of Ca²⁺ binding to calmodulin. Ca²⁺ solution of different concentrations in 100 times diluted PBS buffer were sequentially flowed over the OBL. (c) Equilibrium analysis of Ca²⁺ binding in b. The lighter greyscale curve is fitting of the data, and error bars represent standard deviation at the equilibrium state. (d) Mg²⁺ binding to calmodulin. (e) Equilibrium analysis of Mg²⁺ binding in d. The lighter greyscale curve is fitting of the data, and error bars represent standard deviation at the equilibrium state.

FIGS. 8A-8F. Phosphorylation of SRCtide. (a) The biotinylated SRCtide (substrate) was immobilized to streptavidin OBL. Src kinase (Enzyme) would first combine with the SRCtide to make the enzyme-substrate complex. With the presence of ATP, the phosphorylation peptide was phosphorylated that leads to a charge change of the oscillator. The process can be described by the equation in the figure, where [E], [S], [ES] and [P] are the concentration of enzyme, substrate, enzyme-substrate complex and product. The k₁, k₋₁ and k₂ are the association rate constant, dissociation rate constant and catalytic rate constant, respectively. (b) The raw data obtained with SPR-OBL. The arrow indicates the start point of introducing Src kinase. (c) DC response. Owe to the combination of SRC to the OBL, the surface reflectivity changes leading to an increase in DC response. The solid lines are fitted by the equation (2). (d) The charge of SRCtide OBL is changed due to the phosphorylation, and the solid lines are fitted by the equation (1). (e) (f) Control experiment without the presence of ATP. 4 nm SRC solution is introduced into the system. As expect, SRC will only bind with SRCtide that leads to a DC response (e) without detectable AC response (f).

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, systems, and computer readable media, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Antibody: As used herein, the term “antibody” refers to an immunoglobulin or an antigen-binding domain thereof. The term includes but is not limited to polyclonal, monoclonal, monospecific, polyspecific, non-specific, humanized, human, canonized, canine, felinized, feline, single-chain, chimeric, synthetic, recombinant, hybrid, mutated, grafted, and in vitro generated antibodies. The antibody can include a constant region, or a portion thereof, such as the kappa, lambda, alpha, gamma, delta, epsilon and mu constant region genes. For example, heavy chain constant regions of the various isotypes can be used, including: IgG₁, IgG₂, IgG₃, IgG₄, IgM, IgA₁, IgA₂, IgD, and IgE. By way of example, the light chain constant region can be kappa or lambda. The term “monoclonal antibody” refers to an antibody that displays a single binding specificity and affinity for a particular target, e.g., epitope.

Biomolecule: As used herein, “biomolecule” refers to an organic molecule produced by a living organism. Exemplary biomolecules, include without limitation macromolecules, such as nucleic acids, proteins, peptides, oligomers, carbohydrates, and lipids.

Ligand: As used herein, “ligand” refers to a substance that forms a complex with another molecule, such as a biomolecule.

Nucleic Acid: As used herein, “nucleic acid” refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids can also include nucleotide analogs (e.g., bromodeoxyuridine (BrdU)), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA, cfDNA, ctDNA, or any combination thereof.

Protein: As used herein, “protein” or “polypeptide” refers to a polymer of at least two amino acids attached to one another by a peptide bond. Examples of proteins include enzymes, hormones, antibodies, and fragments thereof.

### DETAILED DESCRIPTION

In some aspects, the present disclosure provides approaches to achieve simultaneous size and charge detection by coupling an SPR sensor with an Oscillating Biomolecular Layer (SPR-OBL). Since conformational change usually accompanies size and/or charge change, SPR-OBL is also sensitive to conformation. In some embodiments, the biolayer is a protein monolayer assembled on top of flexible polymer linkers, which can be driven into oscillation by an applied alternating potential. In some embodiments, binding of ligands alters the refractive index (mass) and the oscillation amplitude (charge) of the biolayer at the same time. In some embodiments, the mass and charge changes are reflected in the DC and AC component of the SPR-OBL signal, respectively, and can be separated using a fast Fourier transform (FFT) filter. In some embodiments, this multi-metric detection capability allows SPR-OBL to measure any interactions/reactions that involve size or charge change (or both), making it particularly useful for measuring small molecule and analyzing enzymatic reactions, which are challenging for traditional label-free techniques. To demonstrate these unique SPR-OBL capabilities, the binding kinetics of various types of binding pairs were measured, including protein-antibody, nanodisc encapsulated membrane protein-small molecule, protein-ion, and enzyme-substrate.

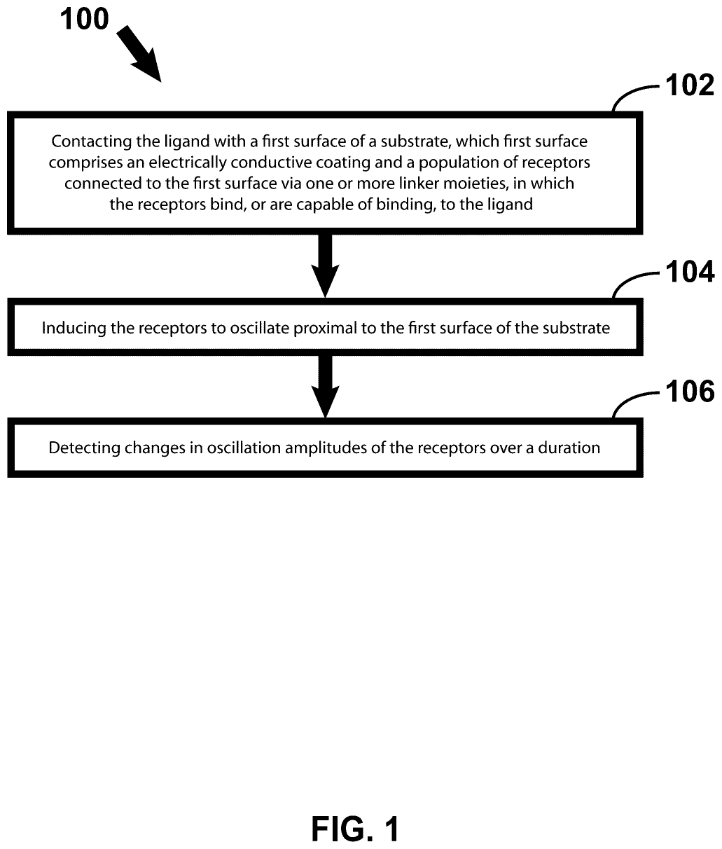

By way of further overview, FIG. 1 is a flow chart that schematically shows exemplary method steps of determining binding kinetics of ligands according to some aspects disclosed herein. As shown, method 100 includes contacting the ligand with a first surface of a substrate, which first surface comprises an electrically conductive coating and a population of receptors connected to the first surface via one or more linker moieties in which the receptors bind, or are capable of binding, to the ligand (step 102). Method 100 also includes inducing the receptors to oscillate proximal to the first surface of the substrate (step 104). In addition, method 100 also includes detecting changes in oscillation amplitudes of the receptors over a duration to thereby determining the binding kinetics of the ligand (step 106).

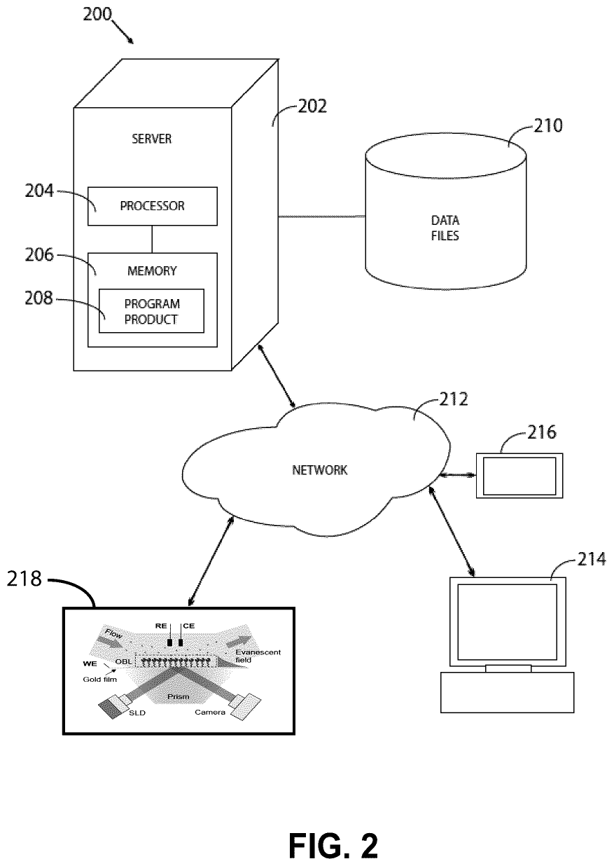

The present disclosure also provides various systems and computer program products or machine readable media. In some aspects, for example, the methods described herein are optionally performed or facilitated at least in part using systems, distributed computing hardware and applications (e.g., cloud computing services), electronic communication networks, communication interfaces, computer program products, machine readable media, electronic storage media, software (e.g., machine-executable code or logic instructions) and/or the like. To illustrate, FIG. 2 provides a schematic diagram of an exemplary system suitable for use with implementing at least aspects of the methods disclosed in this application. As shown, system 200 includes at least one controller or computer, e.g., server 202 (e.g., a search engine server), which includes processor 204 and memory, storage device, or memory component 206, and one or more other communication devices 214, 216, (e.g., client-side computer terminals, telephones, tablets, laptops, other mobile devices, etc. (e.g., for receiving molecular interaction data sets or results, etc.) in communication with the remote server 202, through electronic communication network 212, such as the Internet or other internetwork. Communication devices 214, 216 typically include an electronic display (e.g., an internet enabled computer or the like) in communication with, e.g., server 202 computer over network 212 in which the electronic display comprises a user interface (e.g., a graphical user interface (GUI), a web-based user interface, and/or the like) for displaying results upon implementing the methods described herein. In certain aspects, communication networks also encompass the physical transfer of data from one location to another, for example, using a hard drive, thumb drive, or other data storage mechanism. System 200 also includes program product 208 (e.g., for determining binding kinetics of a ligand as described herein) stored on a computer or machine readable medium, such as, for example, one or more of various types of memory, such as memory 206 of server 202, that is readable by the server 202, to facilitate, for example, a guided search application or other executable by one or more other communication devices, such as 214 (schematically shown as a desktop or personal computer). In some aspects, system 200 optionally also includes at least one database server, such as, for example, server 210 associated with an online website having data stored thereon (e.g., entries corresponding to molecular interaction data, etc.) searchable either directly or through search engine server 202. System 200 optionally also includes one or more other servers positioned remotely from server 202, each of which are optionally associated with one or more database servers 210 located remotely or located local to each of the other servers. The other servers can beneficially provide service to geographically remote users and enhance geographically distributed operations.

As understood by those of ordinary skill in the art, memory 206 of the server 202 optionally includes volatile and/or nonvolatile memory including, for example, RAM, ROM, and magnetic or optical disks, among others. It is also understood by those of ordinary skill in the art that although illustrated as a single server, the illustrated configuration of server 202 is given only by way of example and that other types of servers or computers configured according to various other methodologies or architectures can also be used. Server 202 shown schematically in FIG. 2, represents a server or server cluster or server farm and is not limited to any individual physical server. The server site may be deployed as a server farm or server cluster managed by a server hosting provider. The number of servers and their architecture and configuration may be increased based on usage, demand and capacity requirements for the system 200. As also understood by those of ordinary skill in the art, other user communication devices 214, 216 in these aspects, for example, can be a laptop, desktop, tablet, personal digital assistant (PDA), cell phone, server, or other types of computers. As known and understood by those of ordinary skill in the art, network 212 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 208 is optionally in the form of microcode, programs, cloud computing format, routines, and/or symbolic languages that provide one or more sets of ordered operations that control the functioning of the hardware and direct its operation. Program product 208, according to an exemplary aspect, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 208 implementing the functionality or processes of various aspects of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 208 is optionally copied from the computer-readable medium to a hard disk or a similar intermediate storage medium. When program product 208, or portions thereof, are to be run, it is optionally loaded from their distribution medium, their intermediate storage medium, or the like into the execution memory of one or more computers, configuring the computer(s) to act in accordance with the functionality or method of various aspects disclosed herein. All such operations are well known to those of ordinary skill in the art of, for example, computer systems.

In some aspects, program product 208 includes non-transitory computer-executable instructions which, when executed by electronic processor 204, perform at least: applying an alternating current electric field to a substrate having a first surface and a second surface opposite the first surface, wherein the first surface comprises an electrically conductive coating and a population of receptors connected to the first surface via one or more linker moieties, wherein the receptors bind, or are capable of binding, to a ligand, which alternating current electric field induces the receptors to oscillate proximal to the first surface of the substrate using the power source; introducing an incident light toward the second surface of the substrate from the light source to induce the plasmonic wave at least proximal to the first surface of the substrate; and detecting changes in oscillation amplitudes of the virions over a duration to thereby determine the binding kinetics of the ligand.

In some embodiments, binding kinetics of a ligand is measured using device 218. As shown, device 218 includes a substrate (e.g., gold coated coverglass) having first surface and a second surface opposite the first surface. The first surface comprises an electrically conductive coating (e.g., Au) and a population of receptors connected to the first surface via one or more linker moieties in which the receptors bind, or are capable of binding, to the ligand. Device 218 also includes a power source electrically connected to the substrate. The power source is configured to apply an alternating current electric field to the substrate. Device 218 also includes a prism disposed proximal to the second surface of the substrate. In addition, device 218 also includes a light source configured to introduce light (e.g., collimated light) through the prism to induce a plasmonic wave at least proximal to the first surface of the substrate, and a detector (e.g., a CMOS camera) configured to collect light reflected from the substrate.

### Example: Label-Free Multi-Metric Measurement of Molecular Binding Kinetics by Oscillating Biomolecular Layer

### Introduction

In this example, we present an approach to achieve simultaneous size and charge detection by coupling SPR sensor with an Oscillating Biomolecular Layer (SPR-OBL). Since conformational change usually accompanies size and/or charge change, SPR-OBL is also sensitive to conformation. The biolayer is a protein monolayer assembled on top of flexible polymer linkers, which can be driven into oscillation by an applied alternating potential. Binding of ligands alters the refractive index (mass) and the oscillation amplitude (charge) of the biolayer at the same time. The mass and charge changes are reflected in the DC and AC component of the SPR-OBL signal, respectively, and can be separated using a fast Fourier transform (FFT) filter. This multi-metric detection capability allows SPR-OBL to measure any interactions/reactions that involve size or charge change (or both), making it particularly useful for measuring small molecule and analyzing enzymatic reactions, which are challenging for traditional label-free techniques. To demonstrate these unique SPR-OBL capabilities, we measured the binding kinetics of various types of binding pairs, including protein-antibody, nanodisc encapsulated membrane protein-small molecule, protein-ion, and enzyme-substrate.

Results

Detection Principle

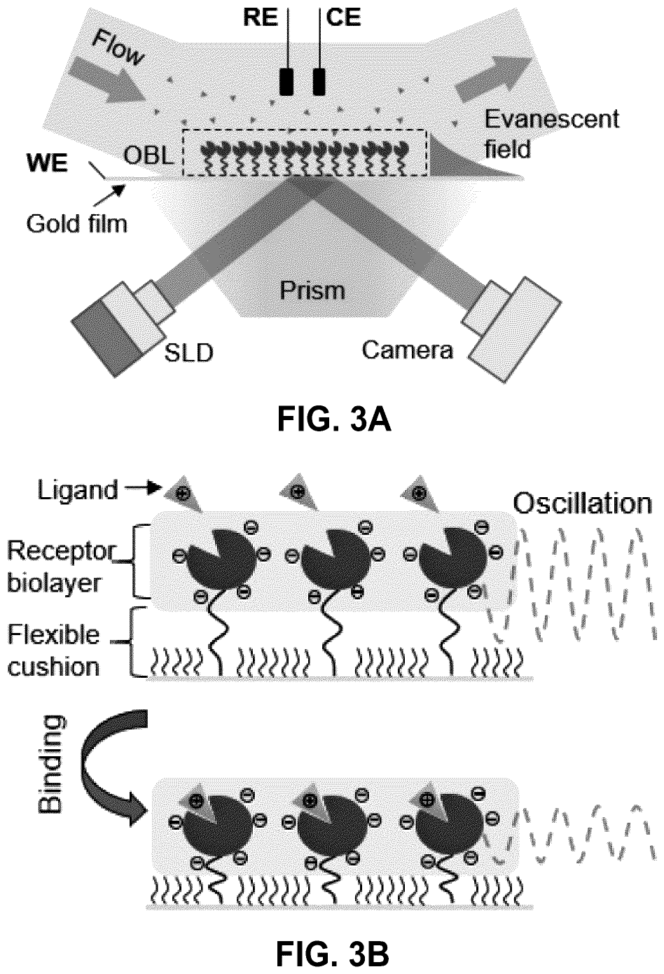

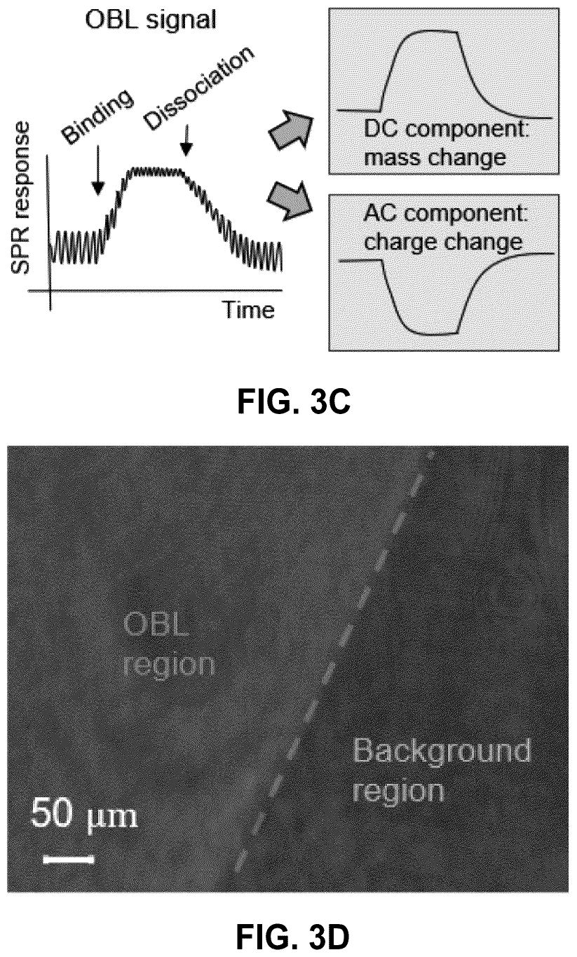

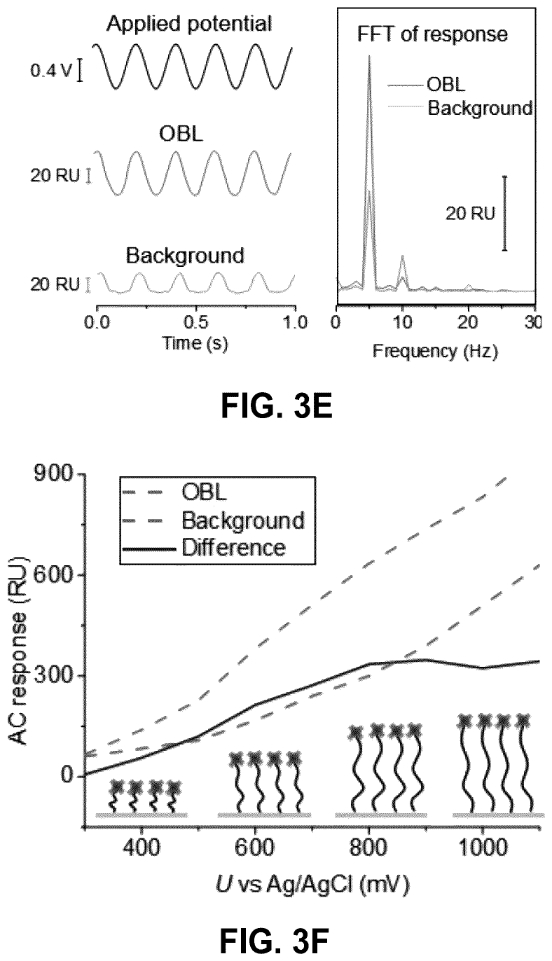

The oscillating biomolecular layer (OBL) is a monolayer of protein, which is connected to the gold film surface by a soft polymer cushion comprising a mixture of long polyethylene glycol (PEG) linkers (molecular weight, 10 kDa) and short PEG spacers (2 kDa or 224 Da) (FIG. 3b). We fabricated a streptavidin OBL to demonstrate the oscillation principle. The gold surface was first modified with SH-PEG10k-biotin and SH-PEG2k at a ratio of 1:10. Then a droplet of streptavidin solution was applied on the PEG cushion to partially functionalized the PEG layer with streptavidin via biotin-streptavidin bonding. The regions with and without streptavidin OBL can be clearly differentiated using SPR microscopy (FIG. 3b). The biolayer is charged in buffer solution, so it can be driven into oscillation by an applied alternating electric field. In contrast, the PEG region (background) cannot oscillate mechanically without the charged protein layer. The nanometer scale oscillation can be precisely measured by SPR owing to the surface confined evanescent field, given by I_{OBL}=I_{OBL,0}×e^{-z/d}, where I_{OBL,0} and I_{OBL} are the SPR intensity when the OBL is at the surface and at a distance of z above the surface, respectively. d is the decay constant of the field, which is about 100 nm.

We applied a 0.4 V, 5 Hz sinusoidal potential (vs. Ag/AgCl) to the gold film surface to modulate the OBL, the SPR response of OBL and background are shown in FIG. 3c. Like the OBL, the background also exhibits oscillating signal but with lower magnitude. This is due to the charging of the electric double layer. The oscillation is in phase with the applied potential because streptavidin is negatively charged: positive potential attracts the streptavidin to the surface and increases the refractive index, vice versa. To extract the oscillation amplitude at 5 Hz, we applied FFT to the oscillation curve in every one second (FIG. 3c). The peak height at 5 Hz in the FFT spectrum was determined as the oscillation amplitude. Next, we studied the electrical response of OBL. By elevating the potential, the oscillation amplitude for both OBL and background increase (FIG. 3d). Because the oscillation signal in OBL region contains both mechanical oscillation and background charging, we subtract the charging signal out from the OBL signal to obtain the mechanical oscillation signal. As shown in FIG. 3d, the net signal increases with potential and eventually reaches a plateau. This is because the PEG linkers approach the maximum extension, consistent with our previous observations on nanoparticle-based oscillators.

When ligand binds to the OBL, the size and/or charge of the OBL is changed, which alters the surface refractive index and/or the oscillation amplitude. FIG. 3e shows a schematic of SPR-OBL signal during a binding detection process, which consists of a DC and an AC component. The DC signal arises from the refractive index change or mass change, similar to conventional SPR; while the AC signal is mainly due to the binding induced charge change on the OBL. The schematic shows an example with obvious mass and charge change. In reality, however, not all interactions have both changes, and as a result, either DC or AC signal is detectable. We will elaborate on this in the next sections below.

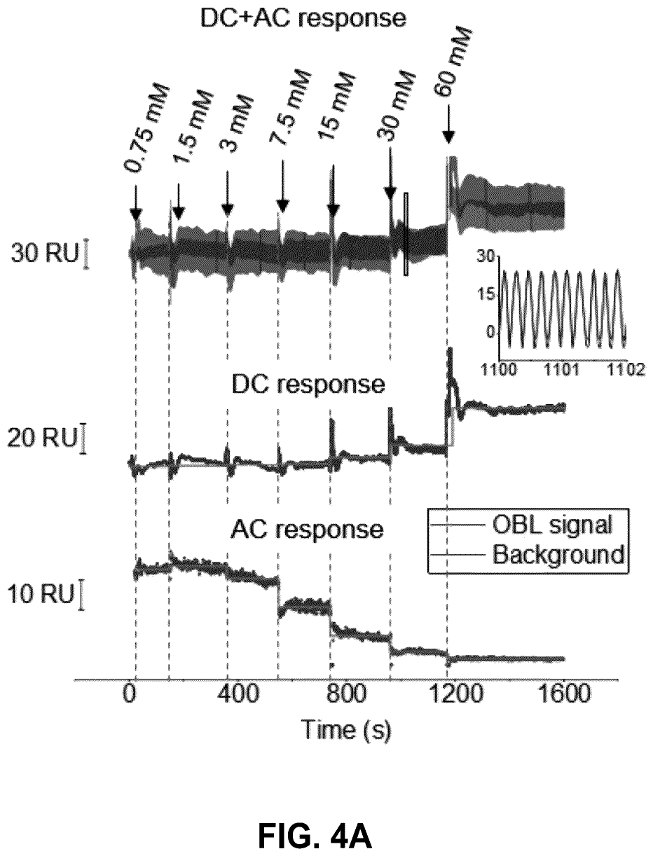

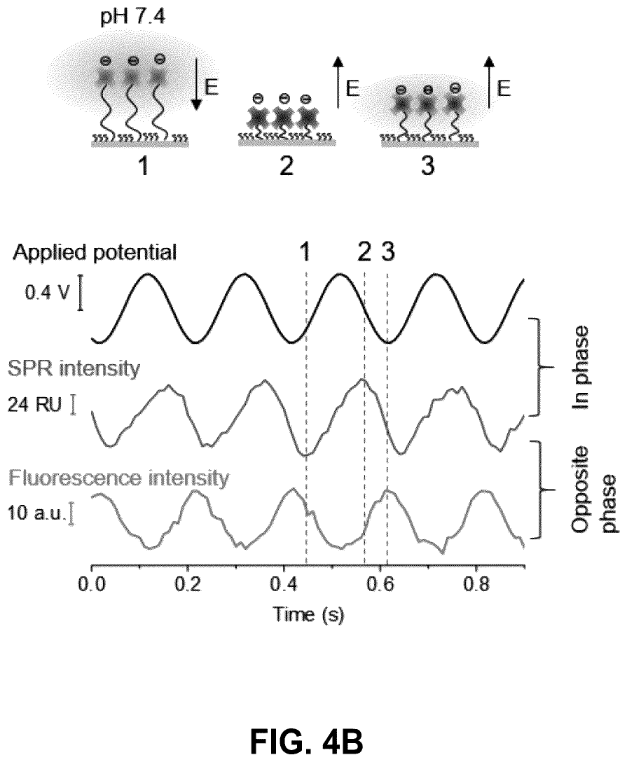

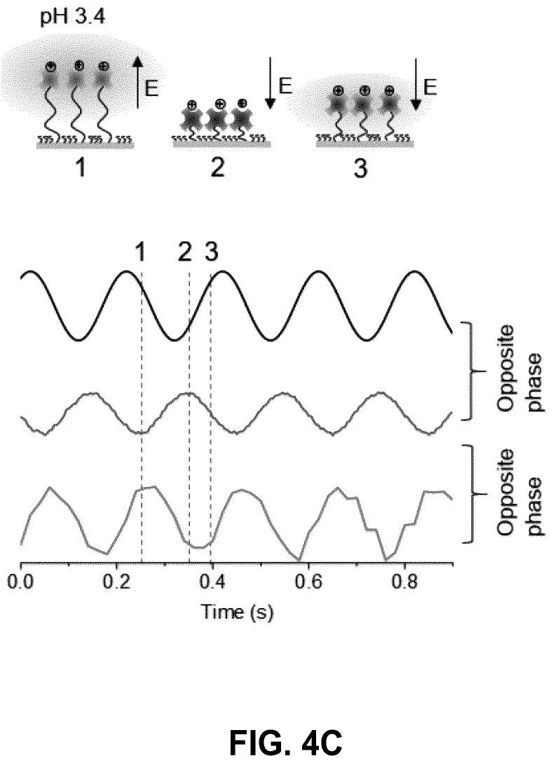

To demonstrate SPR-OBL is capable of probing refractive index and charge at the same time, we measured the response of the aforementioned streptavidin OBL in NaCl solution with different concentrations. We started from a low concentration of 0.75 mM, and then sequentially increased the concentration to 60 mM. The applied potential was maintained at 0.3 V, 5 Hz during the 1600 s detection duration. The response of OBL region and the background region is shown in FIG. 4a. We extracted the DC and AC components for both OBL and background by using a 0.1 Hz low pass filter and a 5 Hz band pass filter, respectively. FIG. 4b shows the DC component of the OBL, which reflects the refractive index change due to salt concentration increase. FIG. 4c presents the AC component of OBL after background subtraction, and the oscillation amplitude reflects the charge on the OBL. The amplitude diminishes as the NaCl concentration increases, because the salt screens the charge on the streptavidin. At 150 mM, about 99% of charge are screened, making the OBL almost neutral. The oscillation amplitude also decreases at extremely low salt concentration, because the solution resistance is high, and the electric field is reduced.

To further validate the OBL signal is caused by mechanical oscillation rather than interfacial charging effects, we fabricated FITC labelled streptavidin OBL and studied the SPR and fluorescence signals with regard to the electrical modulation. When the OBL is driven toward the gold surface, the fluorescence signal is quenched by gold and at the same time the SPR signal reaches the maximum due to increased surface refractive index. On the contrary, the fluorescence recovers and the SPR signal decreases when the OBL is away from the surface (FIG. 4d). Therefore, the phase between the SPR and fluorescence signal should always be opposite. Indeed, our experiment result showed reversed phase between SPR and fluorescence (FIG. 4d). The above experiment was conducted at pH 7.4, and the streptavidin OBL was negatively charged. Hence, the SPR response was almost in phase with the OBL with slight phase shift due to the double layer capacitance. Next, we changed the pH from 7.4 to 3.4, shifting the charge polarity of streptavidin to positive. Again, the phase between SPR and fluorescence was opposite (FIG. 4e), but the phase between SPR and applied potential inversed, which were expected. Moreover, we performed a control experiment by tethering the FITC-streptavidin OBL with a much shorter PEG8 linker (6 nm), and found that both SPR and fluorescence signals diminished significantly. Together, these results indicate that the oscillating signal of OBL is caused by the electrical driven mechanical oscillation of the biolayer rather than surface charging.

Detection of Large Biomolecule

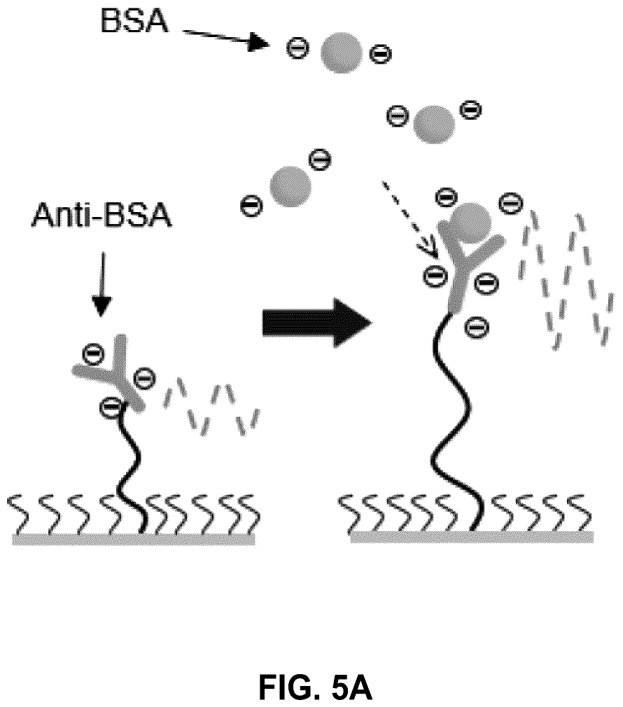

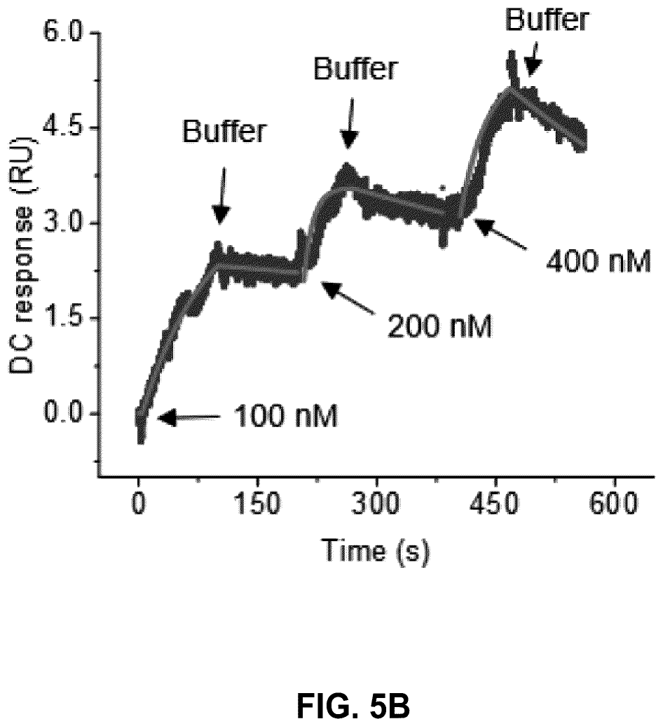

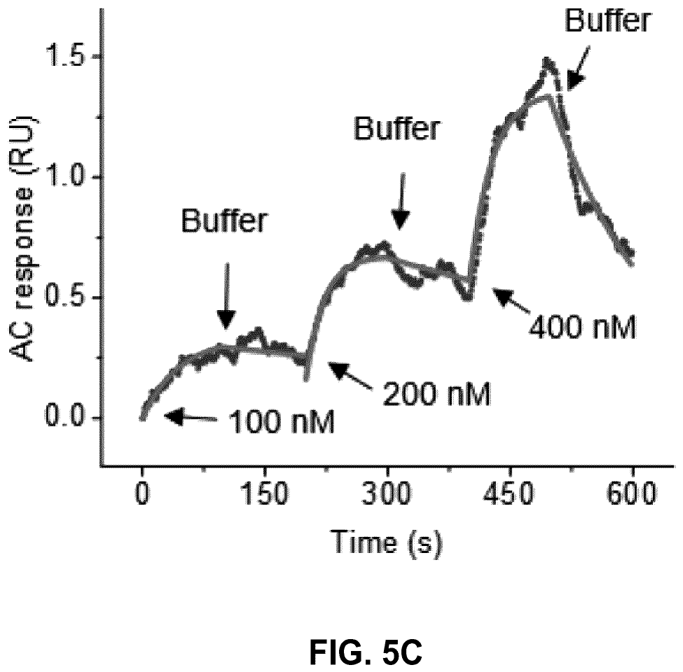

To demonstrate the capability of SPR-OBL in measuring binding kinetics, we first studied the binding of BSA to anti-BSA as it has been well studied by traditional SPR. Anti-BSA was used to fabricate an OBL, and different concentrations of BSA solution were sequentially flown over the surface. The binding of BSA increases the mass and charge of the OBL as shown in FIG. 5a. The DC and AC components of SPR-OBL signal were extracted and plotted in FIGS. 5b and 5c, which represent the mass change and charge change of the binding reaction, respectively. The binding curves were fitted to first-order binding kinetics and the kinetic constants kₐ (association rate constant), k_{d} (dissociation rate constant) and K_{D} (equilibrium constant) were determined. The kₐ, k_{d} and K_{D} for the DC and AC curves are (2.0±0.9)×10⁵ M⁻¹s⁻¹, (1.2±0.4)×10⁻³ s⁻¹ and 13±8 nM, and (1.7±0.4)×10⁵ M⁻¹s⁻¹, (3.4±1.7)×10⁻³ s⁻¹ and 38±25 nM, respectively, which are close to the values in literature. The results show that SPR-OBL is suitable for measuring both mass and charge change in real-time.

Detection of Small Molecule

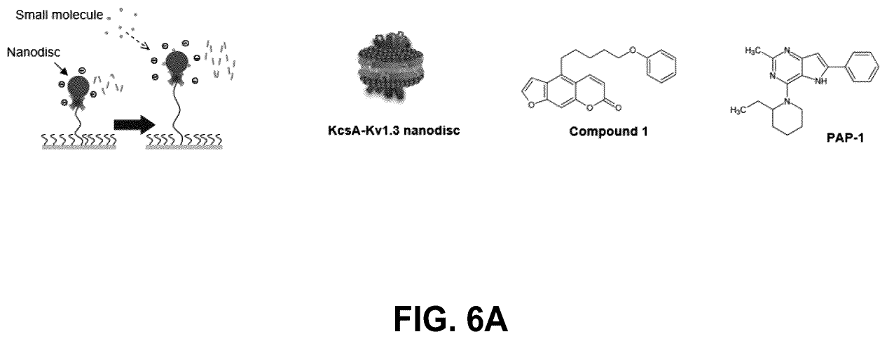

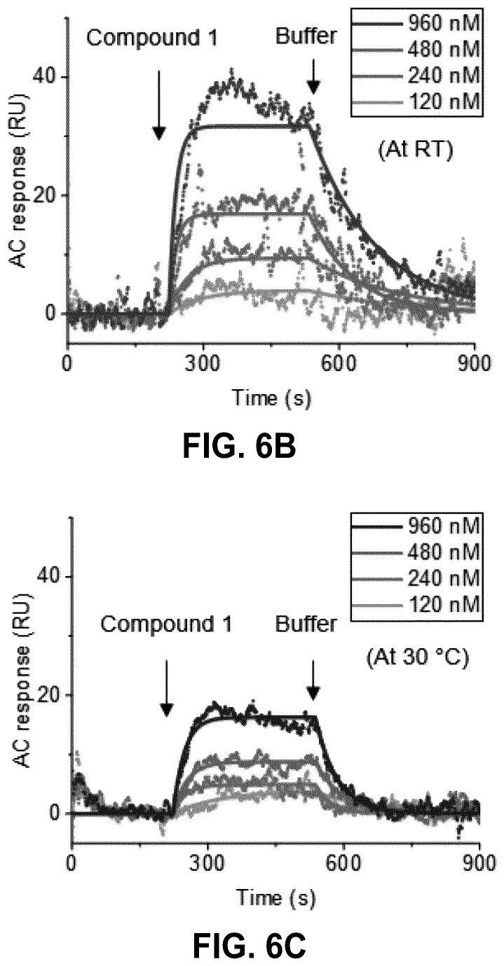

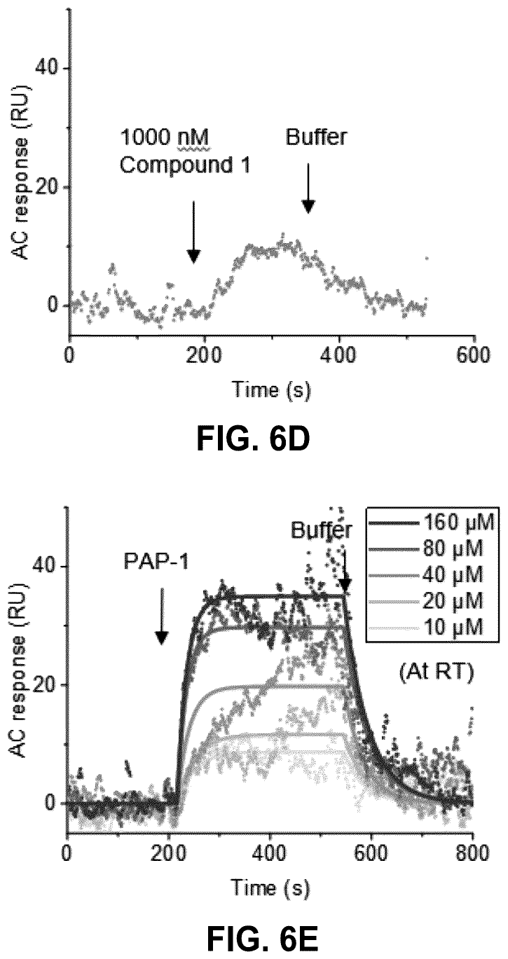

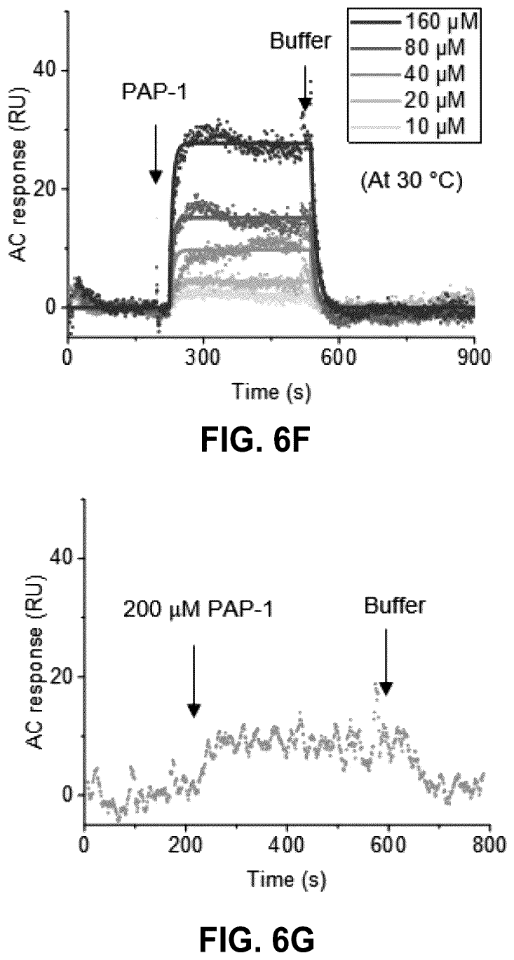

Unlike macromolecules that are inclined to have both mass and charge changes upon binding, small molecules are more likely to have charge change with little mass change. As such, measuring charge could be more sensitive for small molecules. To highlight this advantage of SRR-OBL and its potential for drug screening, we measured two small molecule drug candidates, compound 1 and PAP-1, binding to their target KcsA-Kv1.3, which is a chimeric membrane protein. The KcsA-Kv1.3 was encapsulated in a self-assembled phospholipid bilayer nanodisc that provides a nativelike environment for the membrane proteins to retain the structure and function. Despite the importance of membrane protein as drug targets, measuring the interaction with small molecule drug candidates has been challenging for traditional mass-sensitive technologies, due to the huge mass difference between the small molecule and the membrane protein complex. In our experiment, the biotinylated nanodisc was modified on the streptavidin OBL, as shown in FIG. 6a. Before adding samples, 40 times diluted nanodisc buffer was flowed over the surface for 200 seconds to establish the baseline. The binding of the small molecule to KcsA-Kv1.3 nanodisc caused a charge change on the OBL, which leads to an AC response. As mentioned before, small molecule would only introduce a minor mass change. Therefore, the DC response of OBL is not obvious. By fitting the binding curves of the association and dissociation processes for different concentrations of the small molecules respectively with the first-order kinetics model, the kₐ, k_{d} and K_{D} of compound 1 binding to nanodisc at 25° C. determined to be (8.4±1.1)×10⁴ M⁻¹ s⁻¹, (6.8±1.3)×10⁻³ s⁻¹ and 81±13 nM, as shown in FIG. 6b. According to a previous study that temperature would influence the binding kinetics between KcsA-Kv1.3 and small molecule due to the effect on the flexibility of lipid bilayer, we repeated the experiment under 30° C. and found the kinetic constants became (2.7±0.5)×10⁴ M⁻¹s⁻¹, (1.9±0.4)×10⁻² s⁻¹ and 6.8±1.2×10 2 nM, as shown in FIG. 6c. The results are close to the value in literature. For PAP-1 and KcsA-Kv1.3 nanodisc binding, the kₐ, k_{d} and K_{D} are fitted to be (3.8±0.6)×10² M⁻¹s⁻¹, (2.3±0.1)×10⁻² s⁻¹ and 65±11 μM at 25° C. and (4.8±0.4)×10² M⁻¹s⁻¹, (9.6±1.1)×10⁻² s⁻¹ and (2.1±0.2)×10² μM at 30° C., respectively (FIGS. 6e and 6f). These results proved that temperature indeed influenced the binding kinetics of compound 1 and PAP-1. To verify the binding was specific, we performed control experiments by directly injecting compound 1 and PAP-1 to a streptavidin OBL without nanodisc at 30° C. As shown in FIGS. 6d and 6g, only small response was observed due to nonspecific binding between the small molecules and the streptavidin OBL.

Protein-Ion Interaction

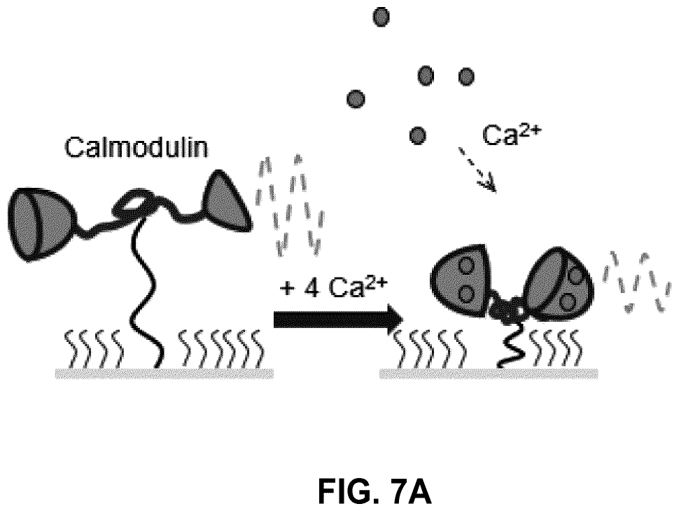

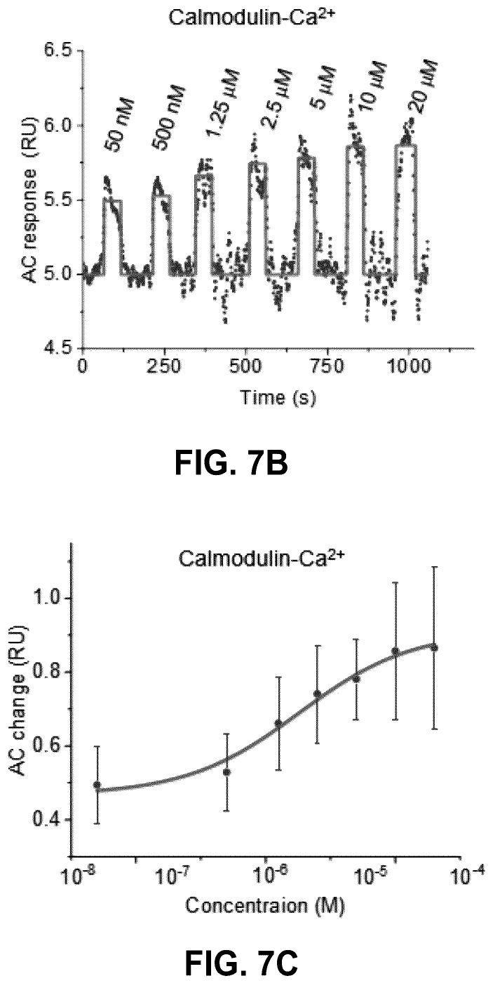

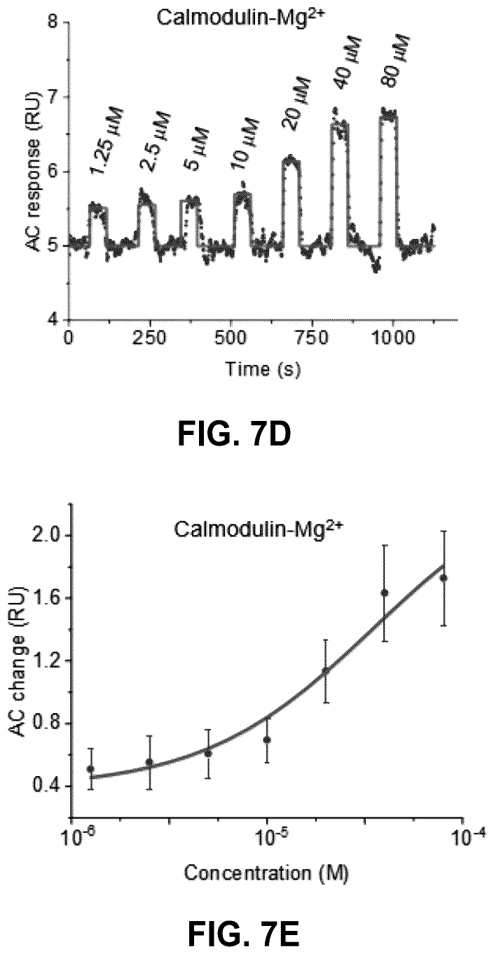

To further demonstrate the high sensitivity of SPR-OBL in small molecule detection, we studied the interaction between calmodulin and calcium ion, whose molecular weight is only 40 Da. Calmodulin is a calcium sensing protein that plays a central role in cell signaling, muscle contraction and cell division. We fabricated a calmodulin OBL and flowed different concentrations of Ca²⁺ over the surface. The binding of Ca²⁺ to calmodulin would lead to charge and conformation change of the OBL but with small mass change (FIG. 7a). The results showed that the binding and unbinding of Ca²⁺ was rapid, and the time resolution of our measurement (1 s) was insufficient to resolve the full kinetics. Thus, we only measured the magnitude of the equilibrium state. The AC response to different Ca²⁺ concentrations were shown in FIG. 7b, and the K_{D} was determined to be 1.9 μM (FIG. 7c), which is close to the value in literature. We note that calmodulin is negatively charged, so Ca²⁺ binding should decrease the amplitude of the AC signal. The increase of AC signal in FIG. 7b is due to artifacts in data processing when correcting the background charging. The raw data has decreased signal but much lower signal-to-noise ratio (not shown). Magnesium would also bind to calmodulin with a lower affinity compared with calcium. Research has found that the binding site for Mg²⁺ could be different from that of Ca²⁺. We measured the interaction between Mg²⁺ and calmodulin as shown in FIGS. 6d and 7e. And the K_{D} was determined as 35 μM, which is about ten times lower than Ca²⁺, in agreement with previous findings.

Phosphorylation Kinetics

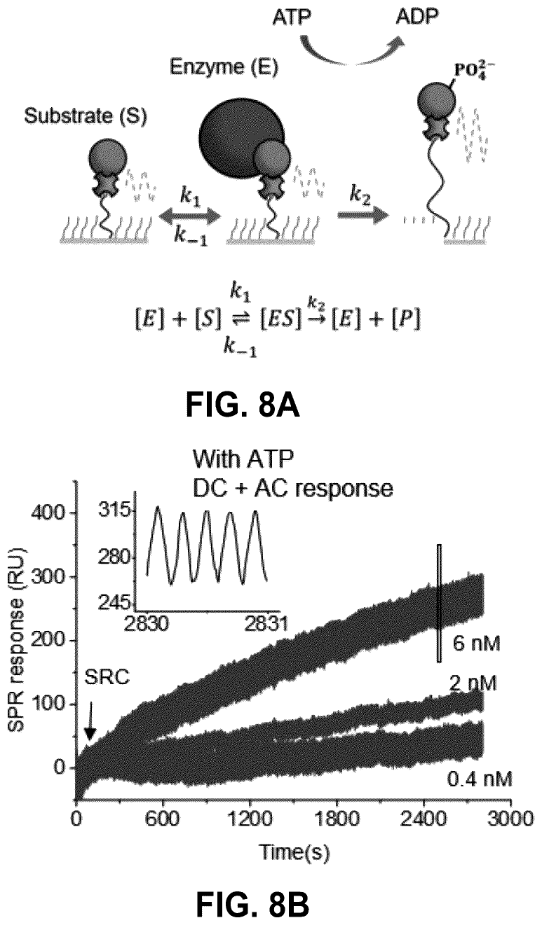

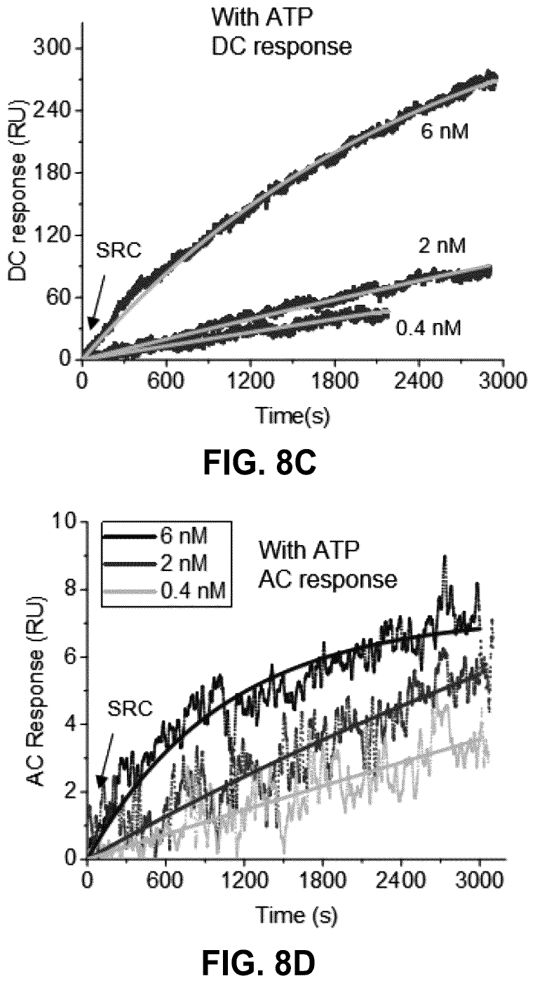

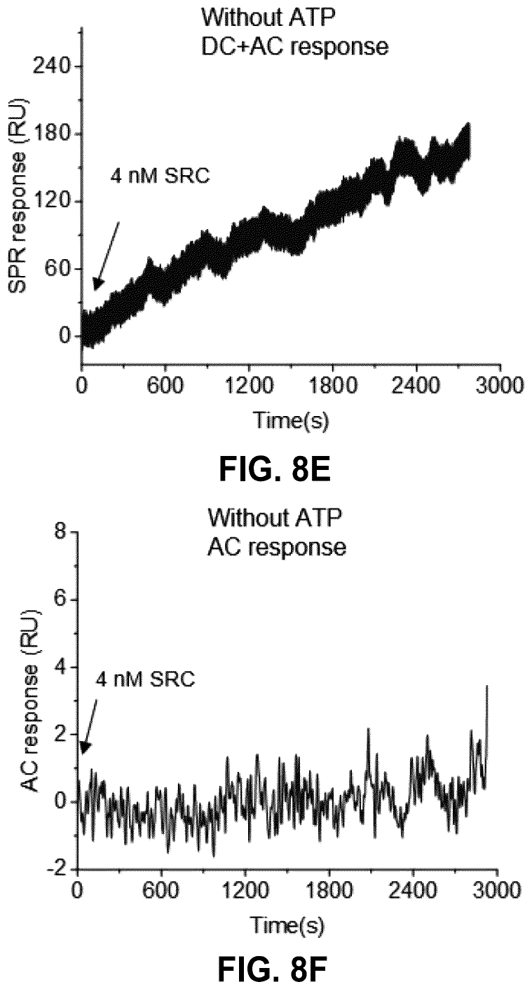

Since SPR-OBL is able to record mass and charge changes at the same time, it is particularly suitable for studying complex enzymatic processes that involve multiple kinetic steps and size and charge changes. Phosphorylation is one of such reactions that includes binding of the kinase to the substrate, adding a phosphate to the substrate, and unbinding of the kinase, where each step accompanies mass or charge change. Despite the importance of phosphorylation in many basic cellular signaling processes and its close relationship with various diseases and cancers, traditional techniques including fluorescence, western blot, and mass spectrometry are unable to resolve the kinetic process in real-time, and each kinetic step needs to be measured separately, limiting the measurement efficiency. In contrast, SPR-OBL can measure multiple steps of the enzyme catalytic reaction in real-time and extract several kinetic parameters in a single measurement. We studied the phosphorylation of SRCtide as an example. SRCtide that was a peptide containing 14 amino acid with the sequence GEEPLYWSFPAKKK (SEQ ID NO: 1) was modified on a streptavidin OBL, which serves as the substrate of the enzyme Src kinase. During the reaction, Src would first bind to the SRCtide, leading to a mass change of the OBL. Meanwhile, the SRCtide is phosphorylated by Src if ATP is present in the system, which induces a charge change. At last, Src unbinds from the phosphorylated substrate (FIG. 8a). The process can be described by

$$E + S {⇌}_{{k}_{- 1}}^{{k}_{1}} ES {\to }^{{k}_{2}} P + E ,$$

where E and S are the enzyme and substrate, ES is the complex, and P is the phosphorylated product, respectively; and k denotes the kinetic constant in each step. We measured the SPR-OBL response of different concentrations of Src in the presence of ATP and the results are shown in FIG. 8b. The DC and AC components were extracted and plotted in FIG. 8c-d. Because the concentration of enzyme is much higher than the surface immobilized substrate, the amount of product generated over time can be described by an enzyme quasi saturable system (EQSS)

$$[ P ] = [ {S}_{0} ] ( 1 - {e}^{\frac{{k}_{2} [ {E}_{0} ]}{{K}_{M} + [ {E}_{0} ]} t} ) \qquad ( 1 )$$

where [E₀], [S₀] and [P] are concentration of SRC, SRCtide and the product, respectively. The Michaelis constant (K_{M}) and catalytic constant (k₂) can be determined to be 4.4 nM and 5.5×10⁻⁴ s⁻¹ by fitting the AC component (FIG. 8d) to Eq. 1. Knowing K_{M} and k₂ the association rate constant (k₁) and dissociation rate constant (k₋₁) can be determined by fitting the DC component (FIG. 8c) to the following equations:

$$[ ES ] = \frac{[ {E}_{0} ] \cdot [ {S}_{0} ]}{{K}_{M} + [ {E}_{0} ]} ( 1 - {e}^{- {k}_{1} ( {K}_{M} + [ {E}_{0} ] ) t} ) \qquad ( 2 )$$

$${K}_{M} = \frac{{k}_{- 1} + {k}_{2}}{{k}_{1}} \qquad ( 3 )$$

We obtain k₁=1.3×10⁴ and k₋₁=2.0×10⁻⁶ s⁻¹, in agreement with literature. To confirm the responses were due to the phosphorylation of SRCtide, a negative control experiment using buffer without ATP was performed. As expected, the enzyme could only bind to the substrate but not phosphorylate the peptide (FIGS. 8e-f). Therefore, we could only get DC response, but no detectable AC response could be observed.

Discussion

Detection Limit and Sensitivity

The mass detection limit is determined by the fluctuation of the baseline, which is 0.74 RU and comparable to most traditional SPR instruments. However, the sensitivity is compromised compared to traditional SPR because of the polymer layer between the sensor surface and the protein layer. The average length of the polymer under electrical modulation is ˜25 nm, considering the decay length of the evanescent field d=100 nm, the sensitivity decreases by a factor of e^{-25/100}=0.78. Also, the protein cannot achieve full coverage on the polymer layer. For example, the coverage of streptavidin is 64%, which further reduces the sensitivity to 0.50. This issue could be alleviated by optimizing the surface chemistry.

The theoretical noise level for charge detection is determined by the FFT spectrum (FIG. 3e), where the standard deviation of the amplitude near 5 Hz about 0.43 RU. The unit RU describes the oscillation amplitude in terms of refractive index (ΔI_{OBL}), which can be converted to electrons using the following equations:

$$Δ {I}_{OBL} = {I}_{OBL , 0} ( 1 - {e}^{- \frac{Δ z}{d}} ) \qquad ( 4 )$$

$$Δ z = \frac{Δ qE}{k} \qquad ( 5 )$$

Where Δz is the oscillation amplitude change in nanometers, Δq is the charge change, E is the electric field (˜2.8×10⁶ V/m), and k is the effective spring constant of the oscillating biolayer (3.6×10⁻⁴ N/m), respectively. Given that ΔI_{OBL}˜0.66 RU and I_{OBL,0}=447 RU, the charge detection limit for a streptavidin OBL is thus estimated to be Δq=0.12 e. However, the noise level of charge is not defined by the detection limit, because the noise is dominated by charge fluctuation, caused by the dynamic adsorption and desorption of ions and charged species in solution to the proteins on the sensor surface. If the surface charge is N e, the fluctuation is in the order of √N e.

The Limitation of Low Ionic Strength

The SPR-OBL measurements were conducted in 100 times diluted PBS or 40 times diluted nanodisc buffer in this work, whose ionic strength were about 1.5 mM and 3.0 mM, respectively. Low ionic strength allows surface charge to be exposed without being affected by the charge screening effect, thus enhances the oscillation and sensitivity. As shown in FIG. 4c, the oscillation amplitude is ˜20 times greater in 1.5 mM NaCl than 60 mM. Simulation shows over 90% charges are maintained at 1.5 mM ionic strength. Nevertheless, such low ionic strength may influence the binding kinetics, especially for densely charged molecules. We measured the interaction between miRNA-21 and its complementary DNA and found that the dissociation was much faster due to the repulsion between negative charges on the nucleic acids. The K_{D} was over 200 times higher than in 1×PBS. Therefore, binding kinetics measured in low ionic strength buffer may be different from physiological condition for highly charged compounds.

Conclusions

SPR-OBL is a label-free detection method for measuring molecular interactions. Beyond traditional SPR, this new technology can detect large biomolecules, small molecules, and ions, as long as either mass, charge, or conformation change is induced by the binding. The multi-metric detection feature also allows to study the kinetics of enzyme catalytic reaction. We anticipate SPR-OBL will expand the capability of SPR with enhanced sensitivity on small molecule detection and molecular charge quantification and serve as a useful tool in biological study and drug screening.

Methods

Materials. The thiol-PEG2k (MW=2 kDa), thiol-PEG10k-bioitin (MW=10 kDa) and thiol-PEG10k-NHS (MW=10 kDa) were purchased from Nanocs Inc. Methyl-PEG₄-thiol (MT(PEG)₄), streptavidin, and FITC-streptavidin were bought from Thermo Fisher Scientific. The active full length SRC protein was purchased from Abcam. SRCtide (sequence: GEEPLYWSFPAKKK (SEQ ID NO: 1)) was purchased from the AnaSpec Inc. BSA, anti-BSA, calmodulin, ATP and 4-(2-hydroxyethyl)-1-piperazineethanesulfonic acid (HEPES) were purchased from Sigma Aldrich. 1× phosphate buffered saline (PBS) was purchased from Corning. Biotinylated KcsA-Kv1.3 nanodisc, compound 1, PAP-1 and standard nanodisc buffer (20 mM Tris, 100 mM NaCl, 0.5 mM EDTA, pH 7.4) were obtained from Amgen.

Fabrication of OBL. The sensor chip (a cover glass coated with 43 nm Au) was rinsed by DI water and ethanol for twice. Then it was annealed by hydrogen flame to further remove surface contaminants. To fabricate the streptavidin OBL, the chip was first dipped in a mixture of 1 mM thiol-PEG2k and 100 μM thiol-PEG10k-bioitin in 1×PBS overnight. The chip was dried with N₂ and a silicone cell was mounted on the chip surface for holding solution. Next, the surface was incubated in 0.2 mg/mL streptavidin in PBS for 30 mins. The functionalized surface was carefully rinsed with PBS for 6 times. Note that the solution should not be completely removed from the cell during the rinsing process, and any air bubbles should be avoided as well, because the tethered protein could be irreversibly absorbed on the surface once exposed to air. The surface coverage of streptavidin on the PEG layer was measured to be 64%, and 46% of the streptavidin could oscillate effectively. The streptavidin OBL was later used to couple biotinylated proteins. To couple KcsA-Kv1.3 nanodisc, 70 μg/mL biotinylated KcsA-Kv1.3 nanodisc in nanodisc buffer was added to the cell and incubated for 30 min. Similarly, the biotinylated SRCtide was immobilized on the streptavidin OBL by incubating in 33 μg/mL SRCtide in kinase buffer containing 5 mM MgCl₂ and 1 mM HEPES (pH 7.4) for 30 mins.

The OBL for proteins without biotin tag was fabricated with a different approach. First, 100 μM thiol-PEG10k-NHS was mixed with protein (16.7 μM calmodulin, 16.7 μM BSA, or 43 μM protein G) in PBS for 2 hours to form a PEG10k-protein complex via NHS-primary amine coupling. Next, the gold surface was dipped in 15 μM MT(PEG)₄ for 3 seconds. This fast process allows the MT(PEG)₄ to be partially modified on the surface, which can reduce the nonspecific adsorption of PEG10k-protein complex. After the solution was removed and the surface was dried, a small droplet (1 μL) of protein complex solution was applied to the surface in the imaging area and incubated for 2 hours. Finally, the whole surface was fully passivated by incubating in 1 mM MT(PEG)₄ for 1 hour.

In the experiment of calcium and magnesium binding to calmodulin, 100 μM thiol-PEG-NHS and 16.7 μM calmodulin were mixed at ratio 1:1 for 2 hours. 15 μM MT(PEG)₄ in 100×PBS was used to modify the surface for 3 seconds, and then add the mixed solution for half an hour. Rinsing the surface with the buffer made of 100×PBS mixed with 10 mM Hepes 6 times, the modified surface was added 15 μM MT(PEG)₄ again for 1 hour. For big molecule modification, 100 uM thiol-PEG-NHS and receptor were mixed for 2 hours. 1 mM thiol-PEG in PBS was used to modify the surface for 3 seconds. Then, the mixed solution was added on the surface for half an hour. Rinsing the surface with 100×PBS 6 times, the modified surface was added 1 mM thiol-PEG again for 1 hour.

Experimental setup. The experiments for oscillation principal validation were carried out on an inverted microscope (Olympus IX-81) with a 60× (NA=1.49) oil immersion objective. A polarized superluminescent light-emitting diode (SLED) (SLD-260-HP-TOW-PD-670, Superlum, Ireland) with a central wavelength of 670 nm and 1 mW power was used as the light source. The electrical modulation was applied by a function generator (33220A, LX1, Agilent) with a potentiostat (AFCBP1, Pine Instrument Company) using a three-electrode configuration, where an Ag/AgCl wire, a Pt coil and the gold surface served as the quasi-reference, counter and working electrode, respectively. A CMOS camera (ORCA-flash 4.0, Hamamstsu) was used to record the plasmonic images. The captured images and applied electrical signal were synchronized by a USB data acquisition card (NI USB-6251, National Instruments). For fluorescence imaging, the light source was the stocking halogen and mercury lamp of the microscope. A filter cube with 488/518 nm for excitation/emission was installed for FITC fluorescence detection.

All the molecular binding experiments were conducted on a commercial prism-based SPR imaging system (SPRm 200, Biosensing Instrument Inc). The electrical modulation was applied using the same equipment as mentioned above. Samples were delivered either by an autosampler with a syringe pump (BI autosampler, Biosensing Instrument Inc.) or a home-built gravity-based drug perfusion system (SF-77B, Warner Instruments). We note that the two delivery methods are similar in terms of binding kinetics detection.

Binding kinetics measurement. All kinetics measurements were performed under a sinusoidal potential with ±0.3 V amplitude (vs Ag/AgCl) and 5 Hz frequency. A gravity-based drug perfusion system was used for the BSA, protein G, phosphorylation, calmodulin, and miRNA measurements at a flow rate of 400 μL/min. Small molecule measurements were performed with the auto-sampler at a flow rate of 150 μL/min. The data was recorded at 100 frames per second.

Signal Processing. The images were recorded and processed by the software of SPRm 200 in real-time. A region of interest (ROI) was selected on the OBL region to record the mean intensity. An adjacent region without OBL could be selected as a reference region to correct the background charging if necessary. The DC component was extracted by a 0.1 Hz low pass fitter, and the AC component was obtained using a 5 Hz FFT filter in every one second which excluded all the other frequencies. The data was smooth-averaged every 10 points before presenting in the figures.

Although this disclosure contains many specific embodiment details, these should not be construed as limitations on the scope of the subject matter or on the scope of what may be claimed, but rather as descriptions of features that may be specific to particular embodiments. Certain features that are described in this disclosure in the context of separate embodiments can also be implemented, in combination, in a single embodiment. Conversely, various features that are described in the context of a single embodiment can also be implemented in multiple embodiments, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular embodiments of the subject matter have been described. Other embodiments, alterations, and permutations of the described embodiments are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example embodiments do not define or constrain this disclosure. Other changes, substitutions, and alterations are also possible without departing from the spirit and scope of this disclosure.

What is claimed is:

1. A method of determining binding kinetics of a ligand, the method comprising:
contacting the ligand with a first surface of a substrate, which first surface comprises an electrically conductive coating and a population of receptors connected to the first surface via one or more linker moieties, wherein the receptors bind, or are capable of binding, to the ligand;
inducing the receptors to oscillate proximal to the first surface of the substrate; and,
detecting changes in oscillation amplitudes of the receptors over a duration, thereby determining the binding kinetics of the ligand.

2. The method of claim 1, wherein the inducing step comprises applying an alternating current electric field to the substrate.

3. The method of claim 1, wherein:
the detecting step comprises separating a detectable signal received from the receptors over the duration into a direct current component and an alternating current component; and/or,
the detecting step comprises introducing an incident light toward a second surface of the substrate to induce a plasmonic wave at least proximal to the first surface of the substrate and detecting a change in intensity of the incident light reflected at an interface of the first surface of the substrate.

4. The method of claim 3, wherein the alternating current electric field is applied to the substrate using an electrode system that comprises a reference electrode, a counter electrode, and a working electrode.

5. The method of claim 1, comprising:
determining a change in mass of one or more of the receptors from a detected reflectivity change of a surface of the substrate;
determining a change in charge of one or more of the receptors from a detected oscillation amplitude change of the one or more of the receptors;
determining size, charge, and/or conformation alterations of one or more of the receptors from the changes in oscillation amplitudes; and/or,
determining the binding kinetics of the ligand in substantially real-time.

6. The method of claim 1, wherein:
the linker moieties comprise polymers;
the linker moieties comprise polyethylene glycol (PEG) moieties and/or biomolecules; and/or,
the receptors comprise a charge.

7. The method of claim 1, comprising:
detecting the changes in the oscillation amplitudes of the receptors using a plasmonic imaging technique and/or a microscopic imaging technique; and/or,
detecting the changes in the oscillation amplitudes of the receptors over the duration using a CMOS camera.

8. The method of claim 1, wherein the electrically conductive coating comprises gold (Au), indium tin oxide (ITO), silver (Ag), copper (Cu), and/or aluminum (Al).

9. The method of claim 1, further comprising one or more spacer moieties connected to the first surface and/or to the linker moieties.

10. The method of claim 1, comprising quantifying the binding kinetics and binding affinity of the ligand using the detected changes in the oscillation amplitudes of the receptors over the duration.

11. The method of claim 1, comprising:
introducing the incident light via at least one objective lens and/or at least one prism; and/or,
introducing the incident light using a superluminescent diode (SLED), a laser and/or a light emitting diode (LED).

12. A receptor oscillator array device, comprising a substrate that comprises a first surface that comprises an electrically conductive coating and a population of receptors connected to the first surface via one or more linker moieties, wherein the receptors bind, or are capable of binding, to a ligand.

13. The receptor oscillator array device of claim 12, wherein the electrically conductive coating comprises gold (Au), indium tin oxide (ITO), silver (Ag), copper (Cu), and/or aluminum (Al).

14. The receptor oscillator array device of claim 12, wherein the linker moieties comprise polyethylene glycol (PEG) moieties and/or biomolecules.

15. The receptor oscillator array device of claim 12, further comprising one or more spacer moieties connected to the first surface and/or to the linker moieties.

16. A system for determining binding kinetics of a ligand, comprising:
a substrate having a first surface and a second surface opposite the first surface, wherein the first surface comprises an electrically conductive coating and a population of receptors connected to the first surface via one or more linker moieties, wherein the receptors bind, or are capable of binding, to the ligand;
a power source electrically connected to the substrate, which power source is configured to apply an alternating current electric field to the substrate;
an objective lens or a prism disposed proximal to the second surface of the substrate;
a light source configured to introduce light through the objective lens or the prism to induce a plasmonic wave at least proximal to the first surface of the substrate;
a detector configured to collect light reflected from the substrate; and
a controller that comprises, or is capable of accessing, computer readable media comprising non-transitory computer-executable instructions which, when executed by at least one electronic processor, perform at least:
applying an alternating current electric field to the substrate to induce the receptors to oscillate proximal to the first surface of the substrate using the power source;
introducing an incident light toward the second surface of the substrate from the light source to induce the plasmonic wave at least proximal to the first surface of the substrate; and,
detecting changes in oscillation amplitudes of the receptors over a duration to thereby determine the binding kinetics of the ligand.

17. The system of claim 16, wherein the electrically conductive coating comprises gold (Au), indium tin oxide (ITO), silver (Ag), copper (Cu), and/or aluminum (Al).

18. The system of claim 16, wherein the linker moieties comprise polyethylene glycol (PEG) moieties and/or biomolecules.

19. The system of claim 16, further comprising one or more spacer moieties connected to the first surface and/or to the linker moieties.

20. The system of claim 16, wherein the non-transitory computer-executable instructions which, when executed by the electronic processor, further perform at least: quantifying the binding kinetics and binding affinity of the ligand using the detected changes in the oscillation amplitudes of the receptors over the duration.


SEQUENCE LISTING (1 sequence)

SEQ ID NO: 1 - protein, 14 aa, synthetic construct
  REGION 1..14: note = Synthetic
GEEPLYWSFPAKKK